(19) United States
(12) Patent Application Publication
(10) Pub. No.: US 2006/0149100 A1
(43) Pub. Date: Jul. 6, 2006

(54) Process for separating and recovering 3-hydroxypropionic acid and acrylic acid

(76) Inventors: Xiangsheng Meng, Chanhassen, MN (US); Paraskevas Tsobanakis, Inver Grove Heights, MN (US); Timothy Abraham, Minnetonka, MN (US)

Correspondence Address: CARGILL, INCORPORATED, LAW/24, 15407 MCGINTY ROAD WEST, WAYZATA, MN, 55391, US

(21) Appl. No.: 10/560,793

(22) PCT Filed: Jun. 25, 2004
(86) PCT No.: PCT/US04/23028

§ 371 Date: Dec. 15, 2005

Related U.S. Application Data
(60) Provisional application No. 60/482,738, filed on Jun. 26, 2003.

(51) Int. Cl. C07C 51/42

(52) U.S. Cl. 562/567

(57) ABSTRACT

Disclosed is a process for separating and recovering 3-hydroxpropionic acid from an aqueous solution comprising 3-hydroxyproprionic acid and acrylic acid, comprising contacting the aqueous solution with an organic extractant, other than ethyl acetate. Also disclosed are processes for separating acrylic acid from aqueous solutions comprising acrylic acid and an organic extractant involving back extraction with water, or distillation in the presence of water. Further disclosed is a process for separating and recovering 3-hydroxyproprionic acid and acrylic acid from an aqueous solution comprising 3-hydroxyproprionic acid and acrylic acid.

### CROSS REFERENCE TO RELATED APPLICATIONS

This application claims the benefit of U.S. Provisional Application No. 60,482,738, filed Jun. 26, 2003.

### FIELD

The invention relates to a process for separating and recovering 3-hydroxypropionic acid from an aqueous solution comprising 3-hydroxypropionic acid, acrylic acid and/or other acid impurities. The aqueous solution may be obtained from any one of multiple preparation routes of 3-hydroxypropionic acid, such as hydration of acrylic acid.

The invention also relates to separation and recovery of acrylic acid from solutions comprising acrylic acid and an organic extractant. There are provided two processes for separating and recovering acrylic acid from the solution. In a first process, a solution comprising acrylic acid and an organic extractant is subjected to back extraction with water to recover the acrylic acid from the extractant. In another process, a solution comprising acrylic acid and an organic extractant having a boiling point lower than 100° C. is distilled in the presence of water to distill the extractant, resulting in an aqueous acrylic acid solution.

Furthermore, the invention includes combining both the process for separating and recovering 3-hydroxypropionic acid and the process for separating and recovering acrylic acid. This allows for the recycling of acrylic acid, and organic extractant, providing economic advantages.

### BACKGROUND

Various methods for separating and recovering 3-hydroxypropionic acid from an aqueous solution comprising 3-hydroxypropionic acid and acrylic acid are known. Included within such methods is distilling acrylic acid from the aqueous solution. Further, it is known that acrylic acid in the aqueous solution is extracted with ethyl acetate.

### SUMMARY

The present invention provides batch and continuous processes for separating and recovering 3-hydroxypropionic acid from an aqueous solution comprising 3-hydroxypropionic acid, acrylic acid and/or other acid impurities by a solvent extraction with any organic extractant, other than ethyl acetate, that allows for separation and recovery of 3-hydroxypropionic acid.

In addition, the present invention provides a process for separating and recovering 3-hydroxypropionic acid in high efficiency, as compared to the use of ethyl acetate, and at high purity.

The present invention also provides batch and continuous processes for recovering acrylic acid and regenerating extractant for reuse, from an acrylic acid-extractant solution. Additionally, the present invention provides a process for separating and recovering 3-hydroxypropionic acid from an aqueous solution comprising 3-hydroxypropionic acid, acrylic acid, and/or other acid impurities, that also includes the separation and recovery of acrylic acid from solutions comprising acrylic acid and an organic extractant.

In accordance with the present invention, it has been found that the above and still further advantages are achieved by extracting acrylic acid and/or other acid impurities from an aqueous solution also comprising 3-hydroxypropionic acid with an organic extractant except ethyl acetate. Acrylic acid may be recovered from the extractant thereby enabling the extractant and acrylic acid to be recycled for reuse. The aqueous solution remaining after acrylic extraction by the extractant comprises 3-hydroxypropionic acid The extractant is organic, and is at least relatively immiscible with an aqueous solution resulting in a separate phase. The extractant is preferably selected from an alcohol, ether, ester (excluding ethyl acetate), ketone, amide, amine, a phosphorus ester, halogenated compound, aromatic compound, phosphine oxide, phosphine sulfide, alkyl sulfide, and mixtures thereof The extraction may be conducted in any manner, for example, in counter current, co-current or cross current extraction system utilizing any equipment such that the separation and recovery of 3-hydroxypropionic acid from acrylic acid can be achieved.

The 3-hydroxypropionic acid recovered by the present process is a reported compound having many applications, and the product herein is useful in such applications. In particular, 3-hydroxypropionic acid is reported as being a useful intermediate in the preparation of various organic materials.

In further embodiment of the present invention, there are provided two processes for separating and recovering acrylic acid from a solution comprising acrylic acid and s organic extractant. A first process comprises subjecting a solution comprising acrylic acid and organic extractant to back extraction with water, using any conventional technique, to separate and recover the acrylic acid from the extractant.

A second process for separating and recovering acrylic acid from a solution comprising acrylic acid and organic extractant where the organic extractant has a boiling point lower than 100° C., comprises distilling the solution, in the presence of water, to distill the organic extractant, thereby resulting in an aqueous acrylic acid solution.

Another embodiment of the present invention comprises combining the process for separating and recovering 3-hydroxypropionic acid from a solution comprising 3-hydroxypropionic acid and acrylic acid with a process for separating and recovering acrylic acid from a solution comprising acrylic acid and an organic extractant. This combined process allows for recovering and recycling acrylic acid and/or extractant, providing economic advantage.

### DETAILED DESCRIPTION

In accordance with the present invention, it has been found that the above features and advantages are achieved by extracting acrylic acid and/or other acid impurities from an aqueous solution also comprising 3-hydroxypropionic acid with an organic extractant. Acrylic acid may be recovered from the organic extractant thereby enabling the organic extractant and acrylic acid to be recycled. The aqueous solution remaining after acrylic acid extraction by the organic extractant comprises 3-hydroxypropionic acid. The extractant is organic, and is at least relatively immiscible with an aqueous solution resulting in a separate phase. The extractant is preferably selected from an alcohol, ether, ester (excluding ethyl acetate), ketone, amide, amine, a phosphorus ester, halogenated compound, aromatic compound, phosphine oxide, phosphine sulfide, alkyl sulfide, and mixtures thereof. The extraction may be conducted in any manner, for example, in counter current, co-current or cross current extraction system utilizing any equipment such that the separation and recovery of 3-hydroxypropionic acid from acrylic acid can be achieved.

In a further embodiment of the present invention, there are provided two processes for separating and recovering acrylic acid from a solution comprising acrylic acid and organic extractant. A first process comprises subjecting a solution comprising acrylic acid and organic extractant to back extraction with water, using any conventional technique, to separate and recover the acrylic acid from the extractant.

A second process for separating and recovering acrylic acid from a solution comprising acrylic acid and organic extractant where the extractant has a boiling point lower than 100° C., comprises distilling the solution, in the presence of water, to distill the extractant, thereby resulting in an aqueous acrylic acid solution.

Another embodiment of the present invention comprises combining the process for separating and recovering 3-hydroxypropionic acid from a solution comprising 3-hydroxypropionic acid and acrylic acid with a process for separating and recovering acrylic acid from a solution comprising acrylic acid and an organic extractant. This combined process allows for recovering and recycling acrylic acid and/or extractant, providing economic advantage.

As reported above, in the process for separating and recovering 3-hydroxypropionic acid herein, the extractant used in the process of the present invention is at least relatively immiscible with an aqueous solution resulting in a separate phase. The extractant used in the present invention is selected preferably from an alcohol, ether, ester (excluding ethyl acetate), ketone, amide, amine, a phosphorous ester, halogenated compound, aromatic compound, phosphine oxide, phosphine sulfide, alkyl sulfide, and mixtures thereof. In more, detail, exemplary extractants suitable for use are described as follows:

Exemplary alcohols suitable for use as extractant have a formula of ROH in which R is C₄-C₂₄ saturated or unsaturated alkyl group, linear or branched, optionally substituted by halogen, alkoxy, amino, alkylamino, hydroxyl groups, cyclic alkyl groups, or a C₆-C₂₄ aryl group, optionally substituted by halogen, alkoxy, amino, alkylamino, or hydroxyl groups. Examples are butanol, amyl alcohol, pentanol, hexanol, heptanol, octanol, decanol, dodecanol, 2-ethyl-1-hexanol, tetradecanol, cyclohexanol, benzyl alcohol, and mixtures thereof.

Exemplary ethers suitable for use as extractant in the present process have the formula R₁OR₂ in which R₁ and R₂ are individually similar or dissimilar, and represent a C₁-C₂₄ saturated or unsaturated alkyl group, linear or branched, optionally substituted by halogen, alkoxy, amino, alkylamino, hydroxyl groups, cyclic alkyl groups, or cyclic ether, or a C₆-C₁₂ aryl group, optionally substituted by halogen, alkoxy, amino, alkylamino, or hydroxyl groups. Examples are diethyl ether, dipropyl ether, diisopropyl ether, dibutyl ether, dihexyl ether, dioctyl ether, methyl t-butyl ether, 2-butoxyethyl acetate, dibutylcarbitol, and mixtures thereof.

Exemplary esters suitable for use as extractant have the formula R₁C(O)OR₂ in which R₁ and R₂ are individually similar or dissimilar, and represent a C₁-C₂₄ saturated or unsaturated alkyl group, linear or branched, optionally substituted by halogen, alkoxy, amino, alkylamino, hydroxyl groups, cyclic alkyl groups or lactones, or a C₆-C₁₂ aryl group, optionally substituted by halogen, alkoxy, amino, alkylamino, or hydroxyl groups. Examples are methyl acrylate, methyl propionate, propyl acetate, isopropyl acetate, butyl acetate, trihexyl trimellitate, trioctyl trimellitate, diethyl butylmalonate, and mixtures thereof.

Exemplary ketones suitable for use as extractant in the present process have the formula R₁C(O)R₂ in which R₁ and R₂ are individually similar or dissimilar, and represent a C₁-C₂₄ saturated or unsaturated alkyl group, linear or branched, optionally substituted by halogen, alkoxy, amino, alkylamino, hydroxyl groups, cyclic alkyl groups or cycloketones, or a C₆-C₁₂ aryl group, optionally substituted by halogen, alkoxy, amino, alkylamino, or hydroxyl groups. Examples are methyl ethyl ketone, methyl isobutyl ketone, cyclohexanone, acetophenone, and mixtures thereof.

Exemplary amides suitable for use as extractant have the formula R₁C(O)NR₂R₃ in which R₁, R₂ and R₃ are individually similar or dissimilar, and represent hydrogen, a C₁-C₂₄ saturated or unsaturated alkyl group, linear or branched, optionally substituted by halogen, alkoxy, amino, alkylamino, hydroxyl groups, or cyclic amides, or a C₆-C₁₂ aryl group, optionally substituted by halogen, alkoxy, amino, alkylamino, or hydroxyl groups. Examples are N,N-dibutyl formamide, N,N-dibutyl acetamide, N,N-dipropyl propionamide, N,N-dibutyl lactamide, 1-octyl-2-pyrrolidinone, 1-dodecyl-2-pyrrolidinone, N,N-diethyl dodecanamide, and mixtures thereof.

Exemplary amines include those having the formula R₁R₂R₃N in which R₁, R₂, and R₃ are individually similar or dissimilar, and represent hydrogen, a C₁-C₂₄ saturated or unsaturated alkyl group, linear or branced, optionally substituted by halogen, alkoxy, amino, alkylamino, or hyroxyl groups, or a C₆-C₁₂ aryl group, optionally substituted by halogen, alkoxy, amino, alkylamino, or hydroxyl groups. Quartenary amine salts may also be used as an extractant. Examples are trioctyl amine, tridecyl amine, tridodecyl amine, and mixtures thereof.

A halogenated compound, phosphorus ester, carbonate ester, phosphine oxide, phosphine sulfide, and alkyl sulfide is also suitable for use as extractant. Exemplary compounds include methylene chloride, chloroform, carbon tetrachloride, 1,2-dichloroethane, trichloroethane, tributyl phosphate, triphenyl phosphate, tritolyl phosphate, dimethyl carbonate, diethyl carbonate, trioctylphosphine oxide, dimethyl methylphosphonate, triisobutyl phosphine sulfide, dihexyl sulfide, diheptyl sulfide, and mixtures thereof.

Any of the extractants may be used alone or in combination with each other. For example, it may be useful to combine an ester extractant with alcohol and/or ether, ketone, amide, halogenated compound, phosphine oxide, phosphine sulfide or alkyl sulfide. In the process for separating and recovering the 3-hydroxypropionic acid by solvent extraction from the solution comprising 3-hydroxypropionic acid and acrylic acid, the extractant for acrylic acid extraction in the organic phase is typically present in an amount of about 1 to about 100 weight percent. The remainder of the component in the organic phase is a saturated or unsaturated hydrocarbon solvent.

The extractions of acrylic acid and/or other acid impurities, from the solution comprising 3-hydroxypropionic acid, is typically carried out at a temperature ranging from about 0° C. to about 100° C., preferably from about 20° C. to about 40° C., and more preferably, from about 20° C. to about 25° C. If pressurized, the extraction may be carried out at a higher temperature, for example, up to about 150° C. The volume ratio of the organic phase to the aqueous phase in the extraction stage ranges from about 20:1 to about 1:20, preferably from about 10:1 to about 1:10, and more preferably from about 5:1 to about 1:5. The extractions may be carried out in accordance with any manner and utilizing any extraction apparatus. The extraction is carried out for any period of time such that the extraction is achieved. For example, the extraction may be carried out in a multistage extraction column, in a counter current co-current or cross current manner.

Remaining after the aqueous phase comprising the 3-hydroxypropionic acid is separated, is an organic phase that comprises acrylic acid and/or other acid impurities, and extractant. In one embodiment for separating and recovering the acrylic acid from the solution comprising acrylic acid and extractant, the solution is back extracted with water. Accordingly, the acrylic acid is recovered from the organic phase, and the extractant is regenerated. The regenerated extractant may be recycled for use in the separation and recovery of the 3-hydroxypropionic acid. The back extraction of the acrylic acid-extractant solution is carried out at a temperature ranging from about 0° C. to about 180° C., preferably from about 50° C. to about 140° C. When the temperature exceeds 100° C., the extraction is typically carried out under pressure. The volume ratio of the organic phase to the aqueous phase ranges from about-20:1 to about 1:20, preferably from about 10:1 to about 1:10, and more preferably from about 5:1 to about 1:5. The back extraction with water is carried out in any manner and with any extraction equipment in any period of time such that the back extraction is achieved. For example, the back extraction may be carried out in a multistage extraction column in counter current, co-current or cross current manner. In another embodiment for separating and recovering acrylic acid from a solution comprising acrylic acid, organic extractant and/or other acid impurities, the organic phase that comprises mainly acrylic acid and/or other acid impurities is subjected to distillation of organic extractant, in the presence of water, for an extractant having a boiling point less than 100° C. The distilled extractant may be recycled back to the extraction for reuse to extract acrylic acid. The distillation of extractant may be carried out, in the presence of water, in accordance with any manner, under any conditions, such that the distillation is achieved. Preferably the distillation temperature is no greater than 100° C. and the pressure is less than or equal to atmospheric pressure. For example, the distillation of extractant may be carried out at any pressure, and at any temperature.

The volume ratio of the organic phase to the aqueous phase in the extraction stage ranges from about 20:1 to about 1:20, preferably from about 10:1 to about 1:10, and more preferably from about 5:1 to about 1:5. The extraction is carried out in accordance with any manner and with any extraction equipment in any period of time such that the extraction is achieved. For example, the extraction may be carried out in a multistage extraction column in counter current, co-current or cross current manner.

The process for separating and recovering 3-hydroxypropionic acid by solvent extraction from a solution comprising 3-hydroxypropionic acid, acrylic acid and/or any other acid impurities, may be combined with any of the processes for separating and recovering acrylic acid from extractant solutions comprising the acrylic acid. The processes may be combined in any manner to provide an economic advantage by allowing recovery and reuse of acrylic acid and extractant.

The invention will be more readily understood by reference to the following examples. There are, of course, many other forms of this invention which will become obvious to one skilled in the art, once the invention has been fully disclosed, and it will accordingly be recognized that these examples are given for the purpose of illustration only, and are not to be construed as limiting the scope of this invention in any way.

### EXAMPLES

In the following Examples, products were analyzed by high pressure liquid chromatography (HPLC), described as follows:

High Pressure Liquid Chromatography (HPLC)

HPLC—the products from the process were analyzed using a Waters 1525 Binary HPLC pump, equipped with a Waters 717 plus Autosampler, and Waters 2410 Refractive Index and Waters 2487 Dual Lambda Absorbance detectors, having a Bio-Rad HP87-H column 0.004 N sulfuric acid as the phase, a flow rate of 0-6 ml/min and a column temperature of 60° C.

### Example 1

In this Example, there were utilized five (5) aqueous stock solutions. The aqueous stock solutions comprise 3-hydroxypropionic acid and acrylic acid. In each of the five aqueous stock solutions, the concentration of 3-hydroxypropionic acid is approximately two times higher than the concentration of the acrylic acid. The aqueous stock solutions are shown in the following Table 1.

TABLE 1
Concentration of acrylic acid and 3-hydroxypropionic
acid in stock solutions.

| Aqueous | Conc. Of acrylic | Conc. Of 3- | Conc. Of total |
|---|---|---|---|
| Stock | acid in stock | hydroxypropionic acid | acids in stock |
| Solution No. | solution, wt. % | in stock solution, wt. # | solution, wt. % |
| 1 | 0.67 | 1.34 | 2.01 |
| 2 | 3.33 | 6.67 | 10.00 |
| 3 | 6.67 | 13.34 | 20.01 |
| 4 | 9.99 | 19.98 | 29.97 |
| 5 | 12.51 | 25.05 | 37.56 |

In carrying out the following extractions of 3-hydroxypropionic acid and acrylic acid from aqueous stock solutions comprising 3-hydroxypropionic acid and acrylic acid, the following organic extractants were used:
a. Decanol
b. Methyl isobutyl ketone (MiBK)
c. Isopropyl ether
d. Methyl acrylate
e. Methyl propionate
f. Methylene chloride (CH₂Cl₂)
g. Toluene
h. Isopropyl acetate
i. Ethyl acetate
j. 50/50 wt % Tributyl phosphate (TBP) and ISOPAR-K isoparaffinic hydrocarbon available from Exxon Mobil Corporation
   The extraction procedure utilized in the Examples herein for separating and recovering 3-hydroxypropionic acid and acrylic acid from aqueous solutions comprising 3-hydroxypropionic acid and acrylic acid is set forth below.

1. To a 15 ml centrifuge tube 5 ml of an acid stock solution and an extractant were added. Masses of the empty centrifuge tube, the aqueous solution and extractant were recorded.

2. The tube was placed on a platform shaker and the contents in the tube were mixed at 230 rpm for 30 minutes at 22° C. (rpm designates revolutions per minute).

3. At the end of mixing, the tube was centrifuged at 4500 rpm for 5 minutes.

4. The volumes of the aqueous and extractant phases in the tube were recorded.

5. The aqueous phase was separated from the extractant phase and the masses of both phases were recorded.

6. The acrylic acid and the 3-hydroxypropionic acid in the aqueous solution were analyzed by HPLC.

7. The concentrations of acrylic acid and 3-hydroxypropionic acid in the organic extractant were calculated by subtracting the concentrations of acrylic acid and 3-hydroxypropionic acid in the aqueous phase from the initial concentration in the stock solution.

The results obtained for the extraction of the aqueous stock solutions described herein to separate and recover acrylic acid and 3-hydroxypropionic acid, utilizing the organic extractants a-j, described above, are reported in the following Table 2.

TABLE 2
Extraction of Acrylic Acid (AA) and 3-Hydroxypropionic Acid (3HP) from
aqueous stock solutions using various extractants.

| Aqueous | | Acid in | | Acid in | | | | |
|---|---|---|---|---|---|---|---|---|
| Stock | | Aqueous | | Extractant | | Partition | | Separation |
| Solution, | | Phase, wt % | | Phase, wt % | | Coefficient, D¹ | | Factor, S² |
| No. | Extractant | AA | 3HP | AA | 3HP | AA | 3HP | AA/3HP |
| 1 | Ethyl | 0.20 | 1.13 | 0.59 | 0.20 | 2.96 | 0.18 | 16.55 |
| 2 | Acetate | 0.97 | 5.77 | 2.87 | 1.21 | 2.97 | 0.21 | 14.18 |
| 3 | | 2.02 | 11.38 | 5.36 | 3.09 | 2.66 | 0.27 | 9.79 |
| 4 | | 3.18 | 17.08 | 7.53 | 5.17 | 2.36 | 0.30 | 7.81 |
| 5 | | 4.22 | 21.16 | 8.07 | 8.68 | 1.91 | 0.41 | 4.66 |
| 1 | TBP/ | 0.13 | 1.15 | 0.55 | 0.23 | 4.23 | 0.20 | 21.15 |
| 2 | ISOPAR-K | 0.88 | 6.52 | 3.32 | 0.52 | 3.80 | 0.08 | 47.27 |
| 3 | hydrocarbon | 2.56 | 13.62 | 5.39 | .01 | 2.39 | 0.07 | 32.12 |
| 4 | | 3.90 | 21.07 | 7.15 | 0.87 | 1.83 | 0.04 | 44.51 |
| 1 | Decanol | 0.31 | 1.37 | 0.47 | 0.03 | 1.51 | 0.02 | 77.18 |
| 5 | | 5.94 | 25.90 | 8.20 | 3.21 | 1.38 | 0.12 | 11.13 |
| 1 | MiBK | 0.23 | 1.32 | 0.59 | 0.03 | 2.51 | 0.02 | 108.82 |
| 5 | | 4.11 | 24.17 | 9.92 | 5.46 | 2.14 | 0.23 | 10.69 |
| 1 | Isopropyl | 0.36 | 1.30 | 0.44 | 0.04 | 1.20 | 0.03 | 40.59 |
| 5 | Ether | 5.92 | 25.72 | 9.79 | 1.85 | 1.65 | 0.07 | 22.91 |
| 1 | Methyl | 0.25 | 1.24 | 0.45 | 0.07 | 1.82 | 0.06 | 31.01 |
| 5 | Acrylate | 4.51 | 21.57 | 8.13 | 6.70 | 1.80 | 0.31 | 5.80 |
| 1 | Methyl | 0.24 | 1.22 | 0.51 | 0.06 | 2.13 | 0.05 | 44.43 |
| 5 | Propionate | 4.54 | 22.63 | 8.55 | 5.54 | 1.88 | 0.25 | 7.69 |
| 1 | CH₂Cl₂ | 0.61 | 1.39 | 0.04 | ND³ | 0.06 | — | — |
| 5 | | 7.84 | 25.05 | 3.72 | 0.53 | 0.48 | 0.02 | 22.45 |
| 1 | Toluene | 0.69 | 1.42 | ND | ND | — | — | — |
| 5 | | 10.57 | 26.72 | 2.47 | ND | 0.23 | — | — |
| 1 | Isopropyl | 0.24 | 1.28 | 0.52 | 0.06 | 2.19 | 0.05 | 43.36 |
| 5 | Acetate | 4.42 | 25.35 | 8.95 | 3.82 | 2.02 | 0.15 | 13.43 |

¹Partition coefficient, D, was calculated by dividing the acid concentration in the extractant phase by the acid concentration in the aqueous phase, for AA and 3HP.
²Separation factor, S, was calculated by dividing the partition coefficient of the acrylic acid by the partition coefficient of 3HP.
³ND means not detectable by HPLC.

The separation factor, S, reported in Table 2 is an indicator of the effectiveness of the separation of 3-hydroxypropionic acid from acrylic acid by the process utilizing an organic extractant. As the value of the separation factor, S, increases, the process is regarded as exhibiting a more effective separation of 3-hydroxypropionic acid from acrylic acid.

A review of the data observed in Table 2 reveals the following conclusions. When extracting aqueous stock solution number 5, that is regarded as a high acid concentration solution, since the total acid concentration was 37.56 weight % the separation factor S, observed when utilizing the process of the present invention vary from 5.8 to 22.91. As a comparison, when utilizing ethyl acetate as an extractant, the separation factor, S, has a value of 4.66. By comparison, and surprisingly and unexpectedly, it has been found that the present process that requires the utilization of specified organic extractants, in achieving the separation and recovery of acrylic acid and 3-hydroxypropionic acid from aqueous solutions comprising acrylic and 3-hydroxypropionic acid, results in a separation factor, S, that is increased by 25% to 491%, relative to a process utilizing ethyl acetate as the organic extractant.

Also observed from the data in Table 2, is the effectiveness of a process for extraction of acrylic acid and 3-hydroxypropionic acid from aqueous solutions comprising 3-hydroxyproprionic acid and acrylic acid, where the aqueous solutions have a low acid concentration, such as 2.01 weight %, in the case of stock solution number 1. In this situation, as in the situation of solutions having a high acid concentration, the separation factor, S, is, surprisingly and unexpectedly, more effective when utilizing a specified organic extractant. More particularly, the data in Table 2 shows that a process for separating and recovering acrylic acid and 3-hydroxypropionic acid from an aqueous solution comprising acrylic acid and 3-hydroxypropionic acid, when utilizing ethyl acetate extractant, has a separation factor, S, of 16.55. As shown in Table 2, the values of the separation factor, S, when using the present process, range from 21.15 to 108.82. When utilizing organic extractants that are within the present invention, as the data in Table 2 shows, the separation factor of the present process exceeds the value obtained when ethyl acetate is used as the organic extractant in the process for separating and recovering acrylic acid and 3-hydroxypropionic acid. Indeed, the extent of the increase in value of the separation factor, S, is surprisingly and unexpectedly, ranging from 28% to 657%.

### Example 2

In this Example, there is shown the process for separating and recovering acrylic acid from a solution comprising acrylic acid and an organic extractant that has a boiling point lower than 100° C. The process involves distillation of the solution, in the presence of water, to distill the organic extractant having a boiling point lower than 100° C., resulting in an aqueous acrylic acid solution.

More particularly, 18.5 grams of isopropyl ether, 3 grams of acrylic acid, and 9 grams of distilled water were introduced into a 100 ml round bottom flask. The organic extractant, isopropyl ether, that has a boiling point of 68° C. at ambient temperature, was then distilled from the solution. The distillation of the isopropyl ether organic extractant, was achieved by applying to the flask, containing the solution of acrylic acid, organic extractant and water, a reduced pressure of about 100 mm Hg, at room temperature (about 20-24° C.). The distillation was completed in about 5 minutes. The resulting isopropyl ether distillate that was collected, and the remaining aqueous solution in the flask, were weighed, and the concentration of acrylic acid in both the isopropyl ether distillate and in the aqueous solution, were determined by means of titration. The amount of acrylic acid that remained in the flask as the aqueous solution was 91%. The amount of acrylic acid that was co-distilled with the isopropyl ether extractant was about 7%. A small amount of water was also co-distilled.

From the data of Example 2, it is apparent that the process for separating and recovering acrylic acid from a solution comprising acrylic acid and an organic extractant that has a boiling point lower than 100° C., comprising distilling the solution in the presence of water, is effective.

### Example 3

Back Extraction of Acrylic Acid with Water at 22° C.

To a 15 ml centrifuge tube, about 3 grams of one of the following acrylic acid (AA) stock solutions and 3 ml of an organic extractant comprising 50/50 wt% tributyl phosphate-ISOPAR-K hydrocarbon were added. The tube was placed on a platform shaker and the contents in the tube were mixed at 230 rpm for 30 minutes at 22° C. After shaking, the tubes were centrifuged at 4500 rpm for five minutes. The aqueous phase was separated from the organic extractant. Both aqueous and organic phases were titrated to determine the concentration of acrylic acid in each phase. The results obtained for the back extraction of acrylic acid with water at 22° C. are shown in the following Table 3.

TABLE 3
Back Extraction of Acrylic Acid with Water from 50/50 wt % tributyl
phosphate-ISOPAR-K hydrocarbon Extractant at 22° C.

| | Stock | | | |
|---|---|---|---|---|
| Sample | Solution | AA in Aqueous | AA in Extractant | Partition |
| No. | AA, wt % | Phase, wt % | Phase, wt % | Coefficient, D¹ |
| 1 | 1.02 | .02 | 1.02 | 5.10 |
| 2 | 4.91 | 1.18 | 4.57 | 3.87 |
| 3 | 9.79 | 2.8 | 7.48 | 2.67 |
| 4 | 14.67 | 5.59 | 10.42 | 1.86 |
| 5 | 19.42 | 8.04 | 12.44 | 1.55 |

¹Partition coefficient, D, was calculated by dividing the acid concentration in the extractant phase by the acid concentration in the aqueous phase, for AA.

### Example 4

Back Extraction of Acrylic Acid with Water at 60° C.

For the back extraction of acrylic acid with water at 60° C., the same experimental procedure as described in Example 3 was used except that the temperature was 60° C. The results obtained were listed in the following Table 4.

TABLE 4
Back Extraction of Acrylic Acid with Water from 50/50 wt % tributyl
phosphate-ISOPAR-K hydrocarbon Extractant at 60° C.

| Back | | | | |
|---|---|---|---|---|
| Extraction | Stock | AA in | AA in | |
| Temperature, | Solution | Aqueous | Extractant | Partition |
| ° C. | AA, wt % | Phase, wt % | Phase, wt % | Coefficient, D |
| 60 | 14.67 | 6.25 | 10.11 | 1.62 |

### Example 5

Back Extraction of Acrylic Acid with Water at 140° C.

For the back extraction with water at 140° C., a Parr pressure reactor was used. To a 100 ml Parr pressure reactor 27.1 grams of 9.82 wt % aqueous solution of acrylic acid and 24.6 grams of an organic extractant comprising 50/50 wt % tributyl phosphate-ISOPAR-K hydrocarbon were added. The reactor was sealed, purged three times with nitrogen gas and then heated to 140° C. The mixture was stirred at 100 rpm for 30 minutes. The mixture was allowed to settle for 2 hours while stirring at 30 rpm. After settling, the samples from the aqueous and organic phases were taken at 140° C. Both phases were titrated for the concentration of acrylic acid. The results obtained are listed in the following Table 5.

TABLE 5
Back Extraction of Acrylic Acid with Water from 50/50 wt % tributyl
phosphate-ISOPAR-K hydrocarbon Extractant at 140° C.

| Back | | | | |
|---|---|---|---|---|
| Extraction | Stock | AA in | AA in | |
| Temperature, | Solution | Aqueous | Extractant | Partition |
| ° C. | AA, wt % | Phase, wt % | Phase, wt % | Coefficient, D |
| 140 | 9.82 | 4.12 | 5.11 | 1.24 |

From the data in Examples 3, 4, and 5, it is observed that back extraction may be carried out, and that acrylic acid and extractant can be recycled. It is further apparent from the data in Examples 3, 4, and 5 that back extraction is preferably carried out at a higher temperature.

### Example 6

An aqueous solution comprising 9.99 wt. % acrylic acid and 19.98 wt. % 3-hydroxypropionic acid is placed in a vessel, and mixed with an equal volume of an organic extractant comprising 50 wt. % tributyl phosphate in ISOPAR-K hydrocarbon. The vessel is placed on a shaker at 230 rpm for 30 minutes at 22° C., and then the mixture is centrifuged at 4500 rpm for 5 minutes. The organic phase is separated from the aqueous phase, and the quantity of acrylic acid and 3-hydroxypropionic acid in each phase is determined by HPLC as previously described. The concentration of acrylic acid and 3-hydroxypropionic acid in the organic extractant is expected to be 7.15 wt. % and 0.87 wt. % respectively. The concentration of acrylic acid and 3-hydroxypropionic acid in the aqueous phase is expected to be 3.90 wt % and 21.07 wt. % respectively.

The above organic phase, containing 7.15 wt % acrylic acid, is placed in a Parr reactor and mixed with an equal weight of distilled water. The reactor is purged several times with nitrogen, and then heated to 140° C. The mixture is stirred at 100 rpm for 30 minutes, and then stirred at 30 rpm for 2 hours to allow the phases to separate. The concentration of acrylic acid in the organic and aqueous phases is determined. The organic phase is expected to comprise approximately 3.72 wt. % of acrylic acid, and the aqueous phase is expected to comprise approximately 2.99 wt % acrylic acid.

Multistage extractions of the initial aqueous solution with organic extractant, and subsequent multistage extractions of the acrylic acid-laden extractant with water is expected to result in almost complete separation of 3-hydroxypropionic acid from acrylic acid. This enables the acrylic acid as well as the extractant to be recycled.

### Example 7

An aqueous solution comprising 12.51 wt. % acrylic acid and 25.05 wt. % 3-hydroxypropionic acid is placed in a vessel, and mixed with an equal volume of Isopropyl ether. The vessel is placed on a shaker at 230 rpm for 30 minutes at 22° C., and then the mixture is centrifuged at 4500 rpm for 5 minutes. The organic phase is separated from the aqueous phase, and the quantity of acrylic acid and 3-hydroxypropionic acid in each phase is determined by HPLC as previously described. The concentration of acrylic acid and 3-hydroxypropionic acid in the organic extractant is expected to be approximately 9.79 wt. % and approximately 1.85 wt. % respectively. Multistage extractions may be performed to achieve almost complete separation of acrylic acid from 3-hydroxypropionic acid.

The above approximately 9.79 wt. % acrylic acid in isopropyl ether is mixed with distilled water (10:3 ratio), and introduced into a flask. The isopropyl ether is removed by distillation at a reduced pressure of approximately 100 mm Hg, and room temperature. The distillation is expected to be complete within a few minutes. The aqueous solution remaining in the flask is expected to contain approximately 25 wt. % acrylic acid. The aqueous acrylic acid and the distilled isopropyl ether can be recycled.

From Examples 6 and 7, it is expected that multistage extraction can give almost complete separation of acrylic acid (AA) and 3-hydroxypropionic acid (3-HP), thus yielding a relatively pure 3-HP product. Further, it is expected that the acrylic acid and extractant can be recycled.

The invention has been described above in detail with particular reference to specific embodiments thereof, but it will be understood that variations and modifications other than as specifically described herein can be effected within the spirit and scope of the invention.

What is claimed is:

1. A process for separating and recovering 3-hydroxypropionic acid from an aqueous solution comprising 3-hydroxypropionic acid and acrylic acid, the process comprising contacting the aqueous solution with an organic phase comprising an organic extractant other than ethyl acetate.

2. The process according to claim 1, wherein the organic extractant is selected from the group consisting of an alcohol, an ether, an ester, a ketone, an amide, a phosphorus ester, a halogenated compound, an aromatic compound, a phosphine oxide, a phosphine sulfide, an alkyl sulfide, and mixtures thereof.

3. The process according to claim 1, wherein the organic extractant is selected from the group consisting of decanol, methyl isobutyl ketone, isopropyl ether, methyl acrylate, methyl propionate, methylene chloride, toluene, isopropyl acetate, tributyl phosphate and mixtures thereof.

4. The process according to claim 1, wherein the organic extractant is present in the organic phase in an amount ranging from about 1 to about 100 weight percent.

5. The process according to claim 1, wherein the process is conducted at a temperature ranging from about 0° C. to about 100° C.

6. The process according to claim 1, wherein the process is conducted at a volume ratio of organic phase to aqueous solution ranging from about 20:1 to about 1:20.

7. The process according to claim 1, wherein the process is a counter current extraction, co-current extraction, or cross current extraction.

8. A process for separating and recovering acrylic acid from an organic phase comprising acrylic acid and an organic extractant, the process comprising contacting the organic phase with an aqueous phase comprising water.

9. The process according to claim 8, wherein the process is conducted at a temperature ranging from about 0° C. to about 180° C.

10. The process according to claim 8, wherein the process is conducted at a volume ratio of organic phase to aqueous phase ranging from about 20:1 to about 1:20.

11. A process for separating and recovering acrylic acid from an organic phase comprising acrylic acid and an organic extractant having a boiling point lower than 100° C., the process comprising heating the organic phase, in the presence of water, to distill the organic extractant.

12. The process according to claim 11, wherein the distillation is conducted at a temperature that is no greater than 100° C. and at a pressure that is less than or equal to atmospheric pressure.

13. The process according to claim 11, wherein the organic extractant is isopropyl ether.

14. A process for separating and recovering 3-hydroxypropionic acid and acrylic acid from an aqueous solution comprising 3-hydroxypropionic acid and acrylic acid, the process comprising the steps of:
a. contacting the aqueous solution with an organic phase comprising an organic extractant other than ethyl acetate, to extract the acrylic acid into the organic phase; and
b. contacting the organic phase formed in step (a) with water to extract the acrylic acid from the organic phase.

15. The process according to claim 14, wherein the organic extractant is selected from the group consisting of an alcohol, an ether, an ester, a ketone, a amide, a phosphorus ester, a halogenated compound, an aromatic compound, a phosphine oxide, a phosphine sulfide, an alkyl sulfide, and mixtures thereof.

16. The process according to claim 14, wherein the organic extractant is present in the organic phase in an amount ranging from about 1 to about 100 weight percent.

17. A process for separating and recovering 3-hydroxyproprionic acid and acrylic acid from an aqueous solution comprising 3-hydroxypropionic acid and acrylic acid comprising the steps of:
a. contacting the aqueous solution with an organic phase comprising an organic extractant, other than ethyl acetate, that has a boiling point lower than about 100° C., to extract the acrylic acid into the organic phase; and
b. heating the organic phase formed is step (a), in the presence of water, to distill off the organic extractant, thereby forming an aqueous acrylic acid solution.
